(19) United States
(12) Patent Application Publication
(10) Pub. No.: US 2005/0002918 A1
(43) Pub. Date: Jan. 6, 2005

(54) Selective treatment of IL-13 expressing tumors

(75) Inventors: Lewis Strauss, Winnetka, IL (US); Raj Puri, Potomac, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignees: NeoPharm, Inc., Lake Forest, IL (US); Gov't of the U.S.A., rep. by the Secretary, Dept. of Health & Human Srvces., Rockville, MD (US)

(21) Appl. No.: 10/842,189

(22) Filed: May 10, 2004

Related U.S. Application Data
(63) Continuation of application No. PCT/US02/36112, filed on Nov. 8, 2002.
(60) Provisional application No. 60/332,477, filed on Nov. 9, 2001.

(51) Int. Cl. A61K 48/00; A61K 38/17

(52) U.S. Cl. 424/93.21; 514/12; 604/500

(57) ABSTRACT

A method of treating tumors that express a receptor for IL-13 is disclosed. The method involves directly introducing into the tumor a cytotoxin that targets the IL-13 receptor. The cytotoxic agent can be introduced by convection-enhanced delivery through a suitable catheter or by other means. Where a convection-enhanced catheter is employed, the method involves positioning the tip of a catheter at least in close proximity to the tumor. After the catheter is positioned, it is connected to a pump which delivers the active agent through the catheter tip to the tumor. A pressure gradient from the tip of the catheter is maintained during infusion.

### FIELD OF THE INVENTION

This invention pertains to a method for selectively treating diseases caused by cells that express IL-13 receptor and particularly to a method of treating solid tumors containing such cells.

### BACKGROUND OF THE INVENTION

Malignant glioma, including glioblastoma multiforme (GBM) and anaplastic astrocytoma (AA) occurs in approximately 17,500 patients annually in the United States. Despite an aggressive multimodal approach to its treatment, no curative therapy is known. Median survival expectation is 9-12 months from diagnosis for GBM and 24-48 months for AA. Despite numerous investigational trials, patients with a recurrence of malignant glioma after initial radiotherapy do not live long.

One approach to eradicating tumor cells is to target cytotoxic agents to the cells. To accomplish this, antibodies or growth factors that bind to cells can be attached to cytotoxic molecules. The binding sites on such cells are known as cell receptors. This method is selective in situations where the targeted receptors are present in substantially higher amounts on target cells than in normal cells. Selectivity is desirable as it minimizes toxicity to normal cells. Exceptionally high levels of the receptor for Interleukin-13 (“IL13R”) have been identified in a number of tumor cells, including malignant gliomas. In contrast, only a few types of normal cells express IL13R and only at low levels. Consequently, IL 13 when combined with a cytotoxic agent has the potential to be a highly effective therapeutic agent for the treatment of IL13R-expressing tumor cells.

To explore the efficacy of such an approach a recombinant fusion protein has been constructed. The fusion protein consists of a truncated bacterial toxin derived from Pseudomonas, PE38QQR, fused to IL13. This agent is more completely described and preliminary cytotoxicity studies can be found in Int. J. Cancer 92, 168-175, which is incorporated herein by reference in its entirety. Unfortunately, when this therapeutic agent is administered systemically, particularly for malignancies in the central nervous system such as malignant gliomas, the drug does not have suitable efficacy.

In general poor overall efficacy of systemic chemotherapy for central nervous system malignancies is attributable to the exclusion of most anti-tumor agents from the brain. Moreover, malignant cells evade treatment by invading brain tissue adjacent to a tumor where they are further sheltered from exposure to any drug that does pass through the blood brain barrier. Thus, even those drugs that do penetrate the blood brain barrier fail to become concentrated in brain tumors and are generally destined to be metabolized and produce undesirable side effects.

New methods are therefore needed that can be used to deliver tumor-targeting drugs directly to tumors, particularly brain tumors, to produce high drug levels within the tumor while minimizing systemic exposure. Ideally such methods will be useful for treating intra-cranial malignancies, such as glioma, in addition to other solid tumors.

The invention provides such a method and composition. These and other advantages of the invention, as well as additional inventive features, will be apparent from the description of the invention provided herein.

### BRIEF SUMMARY OF THE INVENTION

A method of treating tumors that express a receptor for IL-13 is disclosed. The method involves directly introducing into such tumors a cytotoxin that targets the IL-13 receptor. The cytotoxic agent can be introduced by convection-enhanced delivery through a suitable catheter or by other means. Where a convection-enhanced catheter is employed, the method involves positioning the tip of a catheter at least in close proximity to the tumor. After the catheter is positioned, it is connected to a pump which delivers the active agent through the catheter tip to the tumor. A pressure gradient from the tip of the catheter is maintained during infusion.

### DETAILED DESCRIPTION OF THE INVENTION

The present invention is directed to a method for killing a cell that expresses a receptor for interleukin 13 and that is located in a solid tissue comprising, inserting at least one catheter directly into the solid tissue and through the catheter administering a cytotoxic agent to the solid tissue under pressure at a flow rate of about 30 μl/h or more to about 1 ml/h for a predetermined period of time such that a portion of the cytotoxic agent contacts a cell that expresses a receptor for interleukin 13 in the solid tissue and kills the cell.

Any suitable cytotoxic agent that selectively targets tumors that contain cells on which IL-13 receptors reside can be used in practicing the present invention. Such agents typically will have at least two domains, a targeting domain and a cytotoxic domain.

Suitable targeting domains selectively bind the IL-13 receptor and will generally have an affinity constant for the IL-13 receptor that is at least 1/10,000 of the affinity of native IL-13. In addition, targeting domains must maintain their affinity for the IL-13 receptor when joined to the cytotoxic domain. Suitable targeting domains will include for example, IL-13 itself and its derivatives. Suitable IL-13 derivatives include genetically constructed derivatives and chemical derivatives. Genetic derivatives can include truncations, deletions, or mutations so long as a suitable binding affinity for IL-13 receptor is maintained. Similarly, chemical modifications of IL-13 include any chemical modifications that do not preclude binding of the targeting moiety to the IL-13 receptor in the cytotoxin.

Many toxin molecules are known and are suitable for use in the cytotoxic domain. Suitable toxins include pseudomonas exotoxin, ricin, diphtheria toxin, and the like. Suitable cytotoxic domains maintain their cytotoxicity when joined with the targeting domain in the cytotoxin. As with the targeting domain, derivatives of the cytotoxin, including genetic and chemical derivatives are also suitable for use so long as sufficient cytotoxicity is preserved in the ultimate cytotoxin molecule.

The targeting and cytotoxin domains can be joined by any suitable means that provides for retention of the targeting and cytotoxicity characteristics of the cytotoxin. For example, the two domains can be joined chemically such as through cysteine disulfide or other chemical conjugation methods. Desirably, the domains are joined at the the genetic level in a recombinant fusion protein, as is the case with IL13-PE38QQR.

For administration the drug can be dissolved in any suitable pharmaceutical excipient. Suitable excipients include standard solutions of phosphate-buffered saline, normal saline (0.9 wt. %) and preferably 0.2 wt. % human serum albumin in 0.9 wt. % saline.

Any disease caused by cells that express the well known IL-13 receptor can be treated by administration of IL13-PE38QQR. For example, malignant glioblastoma multiforme cells, astrocytoma cells, Kaposi sarcoma cells and renal cell carcinoma among other cells express the IL-13 receptor and can be treated. The method can be used to treat a variety of types of tumors, and is especially useful for treating brain tumors, brain stem tumors, and spinal cord tumors.

Any suitable method for delivering the cytotoxin to the tumor can be used. For example, tumors can be injected with the cytotoxin as through a syringe. Preferably however, the cytotoxin is administered through a catheter by inserting the catheter directly into tissue in the proximity of the tumor. Preferred catheters include those manufactured by Medtronic (e.g., Ventricular #41207, Ventricular #41101, Cardiac/peritoneal #43209, Peritoneal #22014, Peritoneal #22013, #10532, etc.), Phoenix Biomedical Corp (e.g., spiral-port ventricular catheter), and IGN. Other types of catheters (e.g., end-port catheters, side-port catheters, fish-mouth catheters, and the like) also can be employed.

In use, a catheter is joined with a pump that withdraws the cytotoxin from a container and produces enough pressure to cause the drug to flow through the catheter to the tumor cells at controlled rates. Any suitable flow rate can be used such that the tissue is not disrupted or, in the case of brain tissue, the intracranial pressure is maintained at suitable levels so as not to injure the brain tissue. For example flow rates of about 30 μL/h or more to about 1 ml/h are easily tolerated in brain tissue. Catheters for convection-enhanced drug delivery and general methods for administering drugs with such devices are known. See, e.g., U.S. Pat. No. 5,720,720; Am. J. Physiol. 277, R1218-1229; Proc. Nat'l Acad. Sci. (1994) 91, 2076-2080; J. Neurosurg. (1995) 82, 1021-1029. More than a single catheter can be used for the infusion if faster rates than can be achieved with a single catheter are desired. In addition, the treatments can be repeated by reinserting the catheters, if they have been removed, and producing a flow of the cytotoxin to the tumor or tissue around the tumor.

Penetration of the cytotoxin into the tissue is greatly facilitated by positive pressure infusion over a period of days, taking advantage of convection rather than diffusion to aid in drug delivery. This provides for a greater distribution of drug in the treatment area which increases the likelihood that a portion of the drug will come into contact with cells containing IL-13 receptors. When such a contact occurs, the IL-13 targeting domain is thought to bind to the IL-13 receptor. Subsequent to this binding event the cytoxin enters the cell and the toxin domain poisons the cell thereby causing cell death and obliteration of the disease caused by the cell.

Any suitable amount of drug that can be administered in this manner. Suitable amounts are amounts that are effective at retarding the growth of or eradicating the disease causing cells without causing an overabundance of undesirable side effects. For example, with IL13-PE38QQR as little as about 1 μg or more to about 1 mg can be administered in a single treatment. More preferably about 2 μg or more to about 600 μg, even more preferably about 4 μg or more to about 400 μg, and still more preferably about 5 fig or more to about 50 μg is administered.

Tumors can be resected prior to treatment with the drug or, alternatively, tumors can be treated with the drug and then resected. In some case the later procedure may result in the accumulation of necrotic tissue which can be removed. In either situation it is desirable to follow resection with a treatment with the drug so that any disease-causing cells that may have evaded resection and/or the initial drug treatment can be neutralized.

Recent preclinical data demonstrated that the molecular mechanisms of tumor cytotoxicity induced by IL-13PE38QQR includes the induction of apoptosis in tumor cells (Kawakami et al., Mol. Cancer Ther., 1, 999-1007 (2002)). The data that support this observation includes: (a) the time dependent induction of proapoptotic caspases 3, 8 and 9 in tumors treated with IL-13PE38QQR; (b) cleavage of procaspase-3 and poly(ADP-ribose) polymerase (PARP) and; (c) the release of cytochrome C from the mitochondria to the cytosol following injection of IL-13PE38QQR intratumorally. These data demonstrate the mechanisms for the anti-tumor activities of IL-13PE38QQR include the induction of tumor cell apoptosis. The following examples further illustrate the invention but, of course, should not be construed as in any way limiting its scope.

### EXAMPLE 1

This example demonstrates an effective treatment for malignant glioblastoma multiforme. The method takes advantage of a therapeutic agent that targets receptors for interleukin-13 (IL-13R), an immunoregulatory Th2-derived cytokine, on glioblastoma multiforme cells. Interleukin-13 receptors are over-expressed on human glioblastoma cell lines and primary cell cultures. The cytotoxin comprises a fusion protein composed of human IL-13 and a mutated and truncated form of Pseudomonas exotoxin known as PE38QQR. Intratumoral injections of the IL-13 cytotoxin in concentrations of 50 and 100 μg/kg/day for five consecutive days into nude mice having subcutaneous U251 glioblastoma tumors caused a complete response (eradication of the tumor) in 80% and 100% mice, respectively. This response lasted for over eight months after the IL-13 cytotoxin therapy. Three alternate day intratumoral injections of the IL-13 cytotoxin at a dose of 250 μg/kg/day into subcutaneous U87 glioblastoma tumors also produced the same response in all mice.

Intraperitoneal injections of the IL-13 cytotoxin at 25 or 50 μg/kg/dose for five days, twice daily, caused a regression in U251 tumors of about 45% and 58% and caused a complete response in 1 of 5 and 2 of 5 of the treated animals, respectively. A 50 μg/kg intraperitoneal injection into nude mice having U87 xenografts caused a reduction in the tumor burden to one-half. In addition, daily intravenous injections of IL-13 cytotoxin at doses of 25 and 50 μg/kg for five days suppressed the growth of subcutaneous U251 tumors by 75% and 81% and provided a complete response in 1 of 6 animals in each group. The IL-13 cytotoxin therapy manifested no toxicity in any of the treated mice.

IL-13 cytotoxin was also directly injected into glioblastoma multiforme tumors xenografted into the right caudate nucleus of nude rat brain. A single injection of 33.3 μg/kg of IL-13 cytotoxin into intracranial tumors increased median survival by >20% compared to control rats.

### EXAMPLE 2

This example demonstrates the maximum tolerated dose of recombinant ligand-targeted cytotoxin IL13-pseudomonas exotoxin 38QQR (IL13-PE38QQR) that can be delivered by a continuous 96 hour intratumoral infusion in patients with recurrent malignant gliomas. The treatment takes advantage of the high density of IL-13 specific receptors on high-grade glioma specimens. Tissue penetration in the brain of this macromolecule is facilitated by positive pressure infusion, taking advantage of convection. A total of 30 patients in groups of 3-6 were selected based on histologic confirmation of malignant glioma and radiographic evidence of recurrence measuring 1.0 to 5.0 cm in maximum diameter, KPS>60. A stereotaxic biopsy at study entry confirmed the presence of glioma. The IL13-PE38QQR was delivered via 2 intratumoral catheters at a rate of 0.2 ml/hr. The concentration of the IL13-PE38QQR in the infusate was increased in each group. Each patient received 2 treatments 8 weeks apart. Three patients have successfully completed both treatment courses at the starting concentration level of 0.125 μg/ml providing for a dose of 4.8 mg.

### EXAMPLE 3

This example demonstrates positive-pressure microinfusion, also known as convection-enhanced delivery, of IL13-PE38QQR to control malignant glioma. Malignant glioma cells, but not normal brain cells, express IL-13 receptors and are thought to internalize IL 13-PE38QQR toxin, leading to tumor cell death.

This example further demonstrates the histologically-effective concentration (HEC). Tumor biopsy and placement of at least one intratumoral catheter is performed on Day 1, and IL13-PE38QQR infusion is performed over 48 hrs at 400 μL/hr on Day 2-4. The tumor is resected on Day 8, with the goal to accomplish an “en-bloc” resection of the tumor with catheter in place. Tumor tissue is evaluated for evidence of a cytotoxic effect including changes in apoptotic index and proliferation rate, as well as necrosis adjacent to the catheter. Following the resection, two or three catheters are placed into brain adjacent to the tumor resection cavity. Post-resection infusion of 750 μL/hr total for 96 hrs is administered on Days 10-14 to treat any residual surviving glioma that has invaded adjacent brain tissue. Pre-and post-resection infusion starts with IL 13-PE38QQR concentrations of 0.25 μg/mL IL13-PE38QQR.

Pre-operative infusions were well-tolerated in five of six patients tested. In one patient, progressive tumor-related hemiparesis at study entry halted pre-operative drug infusion. In 2 patients, transient changes in affect and cognition were noted during the infusion. All resections and post-resection infusions were well tolerated. One of six patients receiving post-operative infusions at 0.25 μg/mL experienced steroid-responsive hemiparesis with MRI changes one month later. Tumor specimen in one patient after pre-operative IL13-PE38QQR infusion at 0.5 μg/mL reveals regional necrosis in an ovoid zone extending 2-2.5 cm from catheter tip, consistent with drug effect.

Dose limiting toxicity is defined as any Grade 3 or Grade 4 toxicity which is definitely or probably related to study drug. The maximum tolerated dose (“MTD”) is the dose-level below that which causes dose-limiting toxicity in two or more of up to six patients. Geographic necrosis is defined by loss of cellular integrity with eosinophilic staining or by complete cell loss. The finding of greater than about 90% of cells necrotic in the post-infusion specimen, as compared with the pre-infusion biopsy, in a radial distribution at least 2 cm from the catheter tip, demonstrates drug efficacy.

Patients are treated with the following concentrations of the drug: 0.2, 0.5, 1, 2, 3, 4, 6, and 8 by infusing the drug in a pharmaceutically acceptable excipient at a rate of 0.4 ml/h for 48 hours when treated prior to tumor resection. This provides doses of 5, 10, 20, 40, 60, 80, 120, and 150 μg. Post resection treatments with the drug is with identical concentrations administered more aggressively at 0.75 ml/min for 96 hours for total doses of 20, 40, 70, 140, 220, 290, 430, and 580 μg, respectively.

The following Table I demonstrates demographics of six patients:

TABLE I

| | Date of Original | | | | |
|---|---|---|---|---|---|
| | Diagnosis | Age | Sex | KPS | Tumor Site; Pathology |
| Cohort 1 | | | | | |
| Patient 1 | Dec. 18, 2000 | 58 | M | 100 | R temporo-parietal; GBM |
| Patient 2 | Feb. 5, 1997 (AA) | 35 | M | 100 | R temporal; GBM |
| Patient 3 | Sep. 28, 1998 | 33 | F | 100 | R parieto-occipital; GBM |
| Cohort 2 | | | | | |
| Patient 4 | Dec. 1, 1999 | 53 | F | 80 | L fronto-temporo-parietal; GBM |
| Patient 5 | Jan. 21, 1997 | 39 | F | | L fronto-central; GBM |
| Patient 6 | Jan. 7, 2000 | 45 | F | 90 | R fronto-temporal; GBM |

The following Table II demonstrates the toxicity profile and efficacy of the drug treatment when administered prior to tumor resection:

TABLE II

| | Pre-Resection | | |
|---|---|---|---|
| | IL13-PE38QQR | | |
| | Concentration | Toxicities of Pre-Resection | |
| | (μg/mL) | Infusion | Pathology at Resection |
| Cohort 1 | | | |
| Patient 1 | 0.25 | Mildly decreased cognition | No definite necrosis |
| | | during infusion | |
| Patient 2 | 0.25 | Flattened affect & decreased | No definite necrosis |
| | | cognition during infusion | |
| Patient 3 | 0.25 | Transient field cut | No definite necrosis |
| Cohort 2 | | | |
| Patient 4 | 0.5 | None | 2 × 2.5 oval region of |
| | | | necrosis around catheter |
| Patient 5 | 0.5 | Increased R hemiparesis; | Fragmentary; insufficient |
| | | infusion halted | dose |
| Patient 6 | 0.5 | None | Necrosis, but resection |
| | | | suboptimal for anatomy |

Table II shows that 0.25 μm/ml of the drug is infused intratumorally prior to tumor resection, the treatment was well tolerated. When 0.5 μg/ml of the drug was administered the treaetment was well tolerated and demonstrated efficacy as shown by tumor necrosis.

The following Table III demonstrates the toxicity profile and efficacy of the drug treatment when administered after tumor resection:

TABLE III

| | IL13-PE38QQR | Toxicities of | |
|---|---|---|---|
| | Concentration | Post-Resection | |
| | (μg/mL) | Infusion | Surgical Issues |
| Cohort 1 | | | |
| Patient 1 | 0.25 | None | Post-op field cut |
| Patient 2 | 0.25 | None | |
| Patient 3 | 0.25 | None | Only one catheter |
| | | | usable post-op, run |
| | | | at 400 μL/hr |
| Cohort 2 | | | |
| Patient 4 | 0.25 | Transient | Catheter blockage |
| | | severe Rt | delayed post-op |
| | | hemiparesis | infusion by 1 day |
| | | with abnormal | |
| | | MRI at week 5 | |
| Patient 5 | 0.25 | None | |
| Patient 6 | 0.25 | None | |

Table III shows that 0.25 μm/ml of the drug is infused into the situs of the tumor after tumor resection, the treatment was well tolerated. When 0.5 μg/ml of the drug was administered the treaetment was well tolerated and demonstrated efficacy as shown by tumor necrosis.

Table IV shows that when 0.25 lm/ml of the drug is infused into the situs of the tumor after tumor resection, the treatment was well tolerated. When 0.5 μg/ml of the drug was administered the treatment was well tolerated and demonstrated efficacy as shown by tumor necrosis.

TABLE IV

| | Study Entry | Progression-Free | Overall Survival |
|---|---|---|---|
| | Date | Duration (wks) | Duration (wks) |
| Cohort 1 | | | |
| Patient 1 | Jun. 5, 2001 | 22+ | 22+ |
| Patient 2 | Jun. 13, 2001 | 9 | 21+ |
| Patient 3 | Jun. 20, 2001 | pend | 20+ |
| Cohort 2 | | | |
| Patient 4 | Aug. 9, 2001 | 10 | 13+ |
| Patient 5 | Aug. 20, 2001 | pend | 12+ |
| Patient 6 | Aug. 20, 2001 | 11+ | 11+ |

This example has demonstrated that direct intratumoral infusion of IL13-PE38QQR is well tolerated. Direct intratumoral infusion followed by resection is an efficacious treatment for IL-13-expressing brain tumors. IL13-PE38QQR at concentrations of 0.5 μg/mL is cytotoxic for malignant glioma. In addition, post-operative infusion of IL13-PE38QQR into the brain adjacent to resected tumors is well-tolerated such that malignant glioma can be efficaciously treated by direct infusion with IL13-PE38QQR after resection.

### EXAMPLE 4

In preclinical studies, intracerebral injection of IL13-PE38QQR into rat brain was without neurotoxicity at concentrations up to 100 μg/mL. In this trial, the starting concentration is 0.5 μg/mL. Since many glioma cell lines are inhibited at concentrations of 1-10 ng/mL, this regimen could provide a therapeutic dose to tumor.

### EXAMPLE 5

In one clinical glioma study intracerebral injection of IL13-PE38QQR is accomplished using a daily volume of 4.8 mL/catheter (0.2 mL/hr×24 hours), and total infused volume of 38.4 mL/course was held constant. There was a 96 hour infusion at weeks 1 and 9, with the dosing over this period according to the following table:

TABLE V

| Dose level | Dose (μg/ml) | Total Dose (μg) |
|---|---|---|
| 1 | 0.125 | 4.8 |
| 2 | 0.25 | 9.6 |
| 3 | 0.5 | 19.2 |
| 4 | 1.0 | 38.4 |
| 5 | 2.0 | 76.8 |
| 6 | 4.0 | 153.6 |
| 7 | 6.0 | 230.4 |
| 8 | 9.0 | 345.6 |
| 9 | 12.0 | 460.8 |

This study currently is at dose level 4, and data generated to date are presented on the following four pages:

Cohort #1: 0.125 μg/mL for 96 hours, Week 1 and 9

| | | | | | | Radiographic | | |
|---|---|---|---|---|---|---|---|---|
| Patient #; | Date of | | Related AEs | | Related AEs | and/or | | |
| Initials; Age; | Study | Infusion #1 | Grade ≧ 2 and | Infusion #2 | Grade ≧ 2 and | Pathology | Date of | |
| Sex; Dx | Entry | Date; Dose | SAEs: Weeks 1-8 | Date; Dose | SAEs: Weeks 9-17 | Response | Progression | Comments |
| #1001; CT | Nov. | Nov. 28, 2000- | — | Jan. 23, 2001- | — | — | Mar. 15, 2001 | Last Follow-Up: |
| 42yoM; | 27, | Dec. 2, 2000; | | Jan. 27, 2001; | | | PFS: 15 weeks | Sep. 27, 2002 |
| High grade | 2000 | 0.125 μg/mL | | 0.125 μg/mL | | | | Survival: |
| glioma | | | | | | | | 95+ Weeks |
| #1002; RS | Feb. 19, | Feb. 20, 2001- | Abnormal vision; | Apr. 17, 2001- | Brain edema | — | May 28, 2001 | Date of Death: |
| 33yoM; | 2001 | Feb. 24, 2001; | Brain edema | Apr. 21, 2001; | (SAE, Week 14, | | PFS: 14 weeks | Nov. 20, 2001 |
| Malignant | | 0.125 μg/mL | (SAE, Week 1, | 0.125 μg/mL | May 25, 2001); | | | Survival: |
| giloma | | | Feb. 26, 2001); | | Stupor (SAE, | | | 39 weeks |
| | | | Headache; | | Week 14, May | | | |
| | | | Nausea; Vomiting | | 25, 2001) | | | |
| #1003; JC | Mar. 5, | Mar. 6, 2001- | Aphasia; Cranial | May 1, 2001- | Aphasia (SAE, | — | Jun. 28, 2001 | Date of Death: |
| 55yoM; | 2001 | Mar. 10, 2001; | nerve neuropathy; | May 5, 2001; | Week 11, | | PFS: 16 weeks | Jul. 10, 2001 |
| Malignant | | 0.125 μg/mL | Motor neuropathy | 0.125 μg/mL | May 21, 2001); | | | Survival: |
| glioma | | | | | Confusion (SAE, | | | 18 weeks |
| | | | | | Week 11, May 21, | | | |
| | | | | | 2001); | | | |
| | | | | | Hemiparesis | | | |
| | | | | | (SAE, | | | |
| | | | | | Week 11, | | | |
| | | | | | May 21, 2001); | | | |
| #2004; JG | May 25, | May 26, 2001- | Hemiparesis | NA/CR | Heart arrest | Pathologic | — | Date of Death: |
| 51yoM; | 2001 | May 30, 2001; | (SAE, Week 7, | | (SAE, Week 13, | CR at Week 7 | | Aug. 23, 2001 |
| Recurrent | | 0.125 μg/mL | Jul. 10, 2001) | | Aug. 23, 2001); | | | Survival: |
| glioblastoma | | | | | | | | 13 weeks |
| #5005; TW | Jul. 10, | Jul. 13, 2001- | — | NA/PR | — | Radiographic | Mar. 18, 2002 | Last Follow-Up: |
| 41yoM | 2001 | Jul. 17, 2001; | | | | PR at Week 9 | PFS: 35 weeks | Sep. 27, 2002 |
| Malignant | | 0.125 μg/mL | | | | | | Survival: |
| astrocytoma | | | | | | | | 63+ weeks |
| #5006; TD | Oct. 23, | Oct. 25, 2001- | — | NA/PD | — | — | Jan. 7, 2002 | Date of Death: |
| 52yoM | 2001 | Oct. 29, 2001; | | | | | PFS: 10 weeks | Jul. 24, 2002 |
| Recurrent | | 0.125 μg/mL | | | | | | Survival: |
| glioblastoma | | | | | | | | 39 weeks |

Cohort #2: 0.25 μg/mL for 96 hours, Week 1 and 9

| | | | Related AEs | | | Radiographic | | |
|---|---|---|---|---|---|---|---|---|
| Patient #; | Date of | | Grade ≧ 2 | | Related AEs | and/or | | |
| Initials; Age; | Study | Infusion #1 | and SAEs: | Infusion #2 | Grade ≧ 2 and | Pathology | Date of | |
| Sex; Dx | Entry | Date; Dose | Weeks 1-8 | Date; Dose | SAEs: Weeks 9-17 | Response | Progression | Comments |
| #1007; JK | Dec. 18, | Dec. 19, 2001- | Embolus | NA/PD | — | — | Feb. 8, 2002 | Resected on |
| 51yoF; | 2001 | Dec. 23, 2001 | Lower | | | | PFS: 7 weeks | Feb. 8, 2002; |
| Anaplastic | | 0.25 μg/mL | Extremity | | | | | Date of Death: |
| Astrocytoma | | | (SAE, Week | | | | | Feb. 27, 2002 |
| | | | 7, Feb. 2, | | | | | Survival: |
| | | | 2002) | | | | | 10 weeks |
| #2008; MA | Jan. 9, 2002 | Jan. 10, 2002- | Pneumocephaly | Withdrew | Back Pain (SAE, | — | — | Prior SRT |
| 52yoF; | | Jan. 14, 2002 | SAE, Week 2, | Consent | Week 9, Mar. 12, | | | Last Follow-Up: |
| Malignant | | 0.25 μg/mL | Jan. 17, 2002) | | 2002); Motor | | | Sep. 27, 2002 |
| Glioma | | | | | Neuropathy (SAE, | | | Survival: |
| | | | | | Week 9, | | | 37+ weeks |
| | | | | | Mar. 12, 2002) | | | |
| #1009; AS; | Jan. 28, | Jan. 29, 2002- | Aphasia; | NA/PD | — | — | Mar. 26, 2002 | Last Follow-Up: |
| 46yoM; | 2002 | Feb. 2, 2002; | Thrombosis | | | | PFS: 8 weeks | Sep. 27, 2002 |
| Recurrent | | 0.25 μg/mL | (SAE, | | | | | Survival: |
| Glioma | | | Week 7, | | | | | 34+ weeks |
| | | | Mar. 15, 2002) | | | | | |

Cohort #3: 0.5 μg/mL for 96 hours, Week 1 and 9

| | | | | | | Radiographic | | |
|---|---|---|---|---|---|---|---|---|
| Patient #; | Date of | | Related AEs | | Related AEs | and/or | | |
| Initials; Age; | Study | Infusion #1 | Grade ≧ 2 and | Infusion #2 | Grade ≧ 2 and | Pathology | Date of | |
| Sex; Dx | Entry | Date; Dose | SAEs: Weeks 1-8 | Date; Dose | SAEs: Weeks 9-17 | Response | Progression | Comments |
| #2010; DT; | Mar. 22, | Mar. 23, 2002- | Deep | NA/SAE | Infection (SAE, | — | — | Last Follow-Up: |
| 46yoM; | 2002 | Mar. 27, 2002 | Thrombophlebitis | | Week 9, | | | Sep. 27, 2002 |
| Recurrent | | 0.5 μg/mL | (SAE, Week 7, | | May 21, 2002); | | | Survival: |
| Astrocytoma | | | May 9, 2002) | | Deep Vein | | | 27+ weeks |
| | | | | | Thrombosis (SAE, | | | |
| | | | | | Week 9, | | | |
| | | | | | May 21, 2002) | | | |
| #2011; RT; | Mar. 27, | Mar. 28, 2002- | — | NA | Ataxia (SAE, Week | — | — | Last Follow-Up: |
| 67yoM; | 2002 | Apr. 1, 2002 | | | 12, Jun. 18, 2002); | | | Sep. 27, 2002 |
| Recurrent | | 0.5 μg/mL | | | Cerebral Edema | | | Survival: |
| Residual | | | | | (SAE, Week 12, | | | 27+ weeks |
| Malignant | | | | | Jun. 18, 2002); | | | |
| Glioma | | | | | Confusion; | | | |
| | | | | | Hemiparesis (SAE, | | | |
| | | | | | Week 12, | | | |
| | | | | | Jun. 18, 2002); | | | |
| | | | | | Stupor (SAE, Week | | | |
| | | | | | 12, Jun. 18, 2002) | | | |
| #5012; TH; | Apr. 4, | Apr. 5, 2002- | — | Jun. 5, 2002- | Ataxia; | — | Jul. 8, 2002 | Only 1 |
| 43yoM; | 2002 | Apr. 9, 2002 | | Jun. 9, 2002 | Hydrocephalus | | PFS: 13 weeks | Catheter used. |
| Residual | | 0.5 μg/mL | | 0.5 μg/mL | (SAE, Week 17, | | | Last Follow-Up: |
| Recurrent | | | | | Jul. 29, 2002) | | | Sep. 27, 2002 |
| Malignant | | | | | | | | Survival: |
| Glioma | | | | | | | | 25+ weeks |

Cohort #4: 1.0 μg/mL for 96 hours, Week 1 and 9

| | | | | | | Radiographic | | |
|---|---|---|---|---|---|---|---|---|
| Patient #; | Date of | | Related AEs | | Related AEs | and/or | | |
| Initials; Age; | Study | Infusion #1 | Grade ≧ 2 and | Infusion #2 | Grade ≧ 2 and | Pathology | Date of | |
| Sex; Dx | Entry | Date; Dose | SAEs: Weeks 1-8 | Date; Dose | SAEs: Weeks 9-17 | Response | Progression | Comments |
| #3013; AR; | Jul. 15, | Jul. 16, 2002- | Hallucinations; | Sep. 10, 2002- | — | — | — | Last Follow-Up: |
| 31yoM; | 2002 | Jul. 20, 2002 | Headache; | Sep. 14, 2002 | | | | Sep. 27, 2002 |
| GBM | | 1.0 μg/mL | | 1.0 μg/mL | | | | Survival |
| | | | | | | | | 10+ weeks |

### EXAMPLE 6

In another clinical glioma study intracerebral injection of IL13-PE38QQR is accomplished using a 48 hour infusion of 400 μL/hour), starting one week prior to tumor resection, and a 96 hour infusion (750 μL/hour) was begun two days after tumor resection. The treatment was run in three stages as follows:

Stage 1

| Dosage | Pre-Resection | | Post-Resection | |
|---|---|---|---|---|
| level | Dose (μg/ml) | Total dose (μg) | Dose (μg/ml) | Total dose (μg) |
| 1 | 0.25 | 4.8 | 0.25 | 18.0 |
| 2 | 0.5 | 9.6 | 0.25 | 18.0 |
| 3 | 1.0 | 19.2 | 0.25 | 18.0 |
| 4 | 2.0 | 38.4 | 0.25 | 18.0 |

| | | |
|---|---|---|
| Dosage level | Dose (μg/ml) | Total dose (μg) |

Stage 2 (Post Resection)

| | | |
|---|---|---|
| 1 | 0.5 | 36.0 |
| 2 | 1.0 | 72.0 |
| 3 | 2.0 | 144.0 |

Stage 3 (Post Resection)

| | | |
|---|---|---|
| 1 | 5 | 90 |
| 2 | 6 | 108 |
| 3 | 7 | 126 |

This study currently is at dose level 1 of Stage Two, and data generated to date ed on the following five pages:

Cohort #1: 0.25 μg/mL Pre-Resection; 0.25 μg/mL Post-Resection

| | | | | Related | | | Related | | |
|---|---|---|---|---|---|---|---|---|---|
| | | | | AEs | | | AEs | | |
| Patient #; | | Biopsy/ | | Grade ≧ 2 | | | Grade ≧ 2 | | |
| Initials; Age; | Date of | Catheter | Infusion #1 | and SAEs: | | Infusion #2 | and SAEs: | Date of | |
| Sex; Dx | Diagnosis | Date | Date; Dose | Infusion 1 | Pathology | Date; Dose | Infusion 2 | Progression | Comments |
| #101; DMH | Dec. 8, | Jun. 5, | Jun. 6, 2001- | Headache | No evidence | Jun. 14, 2001- | Fatigue | — | Mild hyponatremia; |
| 58yoM; | 2000 | 2001 | Jun. 8, 2001; | | of necrosis | Jun. 18, 2001; | | | visual deficit post-op |
| R-temporal | | | 0.25 μg | | | 0.25 μg | | | Last Follow-up: |
| GBM | | | | | | | | | Sep. 27, 2002 |
| | | | | | | | | | Progression Free |
| | | | | | | | | | Survival: 68+ Weeks |
| #102; HJM | Feb. 5, | Jun. 13, | Jun. 14, 2001- | Confusion | No evidence | Jun. 22, 2001- | Brain | Aug. 15, 2001 | Partial seizures |
| 35yoM; | 1997 | 2001 | Jun. 16, 2001; | | of necrosis | Jun. 26, 2001; | edema; | PFS: 9 Weeks | increased; |
| R-temporal | [initial | | 0.25 μg | | | 0.25 μg | Headache; | | quadrantanopia; |
| GBM | AA] | | | | | | Pain | | Progressive Disease |
| | | | | | | | | | Died: Mar. 13, 2002 |
| | | | | | | | | | Survival: 39 Weeks |
| #103; CGM | Sep. 28, | Jun. 20, | Jun. 21, 2001- | — | No evidence | Jun. 29, 2001- | — | Sep. 26, 2001 | Hemiparesis; |
| 33yoF; | 1998 | 2001 | Jun. 23, 2001; | | of necrosis | Jul. 3, 2001; | | PFS: 14 Weeks | Paresthesia; |
| R-parieto- | | | 0.25 μg | | | 0.25 μg | | | Post-op: 1 catheter |
| occipital GBM | | | | | | | | | used, was run at |
| | | | | | | | | | 400 μL/hr; ? PD vs. |
| | | | | | | | | | translent |
| | | | | | | | | | enhancement |
| | | | | | | | | | Last Follow-up: |
| | | | | | | | | | Sep. 27, 2002 |
| | | | | | | | | | Survival: |
| | | | | | | | | | 62+ Weeks |

Cohort #2: 0.5 μg/mL Pre-Resection; 0.25 μg/mL Post-Resection

| | | | | Related AEs | |
|---|---|---|---|---|---|
| Patient #; | | Biopsy/ | | Grade ≧ 2 | |
| Initials; Age; | Date of | Catheter | Infusion #1 | and SAEs: | |
| Sex; Dx | Diagnosis | Date | Date; Dose | Infusion 1 | Pathology |
| #201; JAR; | Nov. 30, | Aug. 9, | Aug. 10, 2001- | — | 2 × 2.5 cm |
| 53yoF; | 1999 | 2001 | Aug. 12, 2001; | | oval necrosis |
| L Fronto- | | | 0.5 μg | | |
| tempor-parietal | | | | | |
| GBM | | | | | |
| #202; NWC; | Dec. 1, | Aug. 20, | Aug. 21, 2001- | — | Suboptimal |
| 45yoF; | 1999 | 2001 | Aug. 23, 2001; | | specimen w/ |
| R Fronto- | | | 0.5 μg | | necrosis |
| temporal GBM | | | | | |
| #104; TMW | Jan. 22, | Aug. 20, | Aug. 22, 2001- | Headache; | Fragmentary |
| 38yoF; | 1997 | 2001 | Aug. 23, 2001; | Hemiparesis | |
| L Fronto- | | | 0.5 μg | (SAE, before | |
| parietal GBM | | | Insufficient | first infusion, | |
| | | | dose, | Aug. 21, 2001); | |
| | | | replaced in | Speech | |
| | | | enrollment | disorder | |
| #105; S-C | Oct. 16, | Oct. 30, | Oct. 31, 2001- | Headache | — |
| 51yoF; | 2000 | 2001 | Nov. 2, 2001; | | |
| GBM | | | 0.5 μg | | |
| #203; JWS | Dec. 14, | Nov. 5, | Nov. 6, 2001- | — | Major (>75%) |
| 46yoM; | 1999 | 2001 | Nov. 8, 2001; | | necrosis 1 cm |
| GBM | | | 0.5 μg | | from tip |

| Patient #; | | Related AEs | | |
|---|---|---|---|---|
| Initials; Age; | Infusion #2 | Grade ≧ 2 and | Date of | |
| Sex; Dx | Date; Dose | SAEs: Infusion 2 | Progression | Comments |
| #201; JAR; | Aug. 18, 2001- | Hemiparesis | Oct. 16, 2001 | Catheter kinking delayed |
| 53yoF; | Aug. 23, 2001; | (SAE, Sep. 11, | PFS: 9 Weeks | post-op infusion; |
| L Fronto- | 0.25 μg | 2001); Seizure | | Died Jan. 3, 2002 |
| tempor-parietal | | (SAE, Sep. 11, | | Survival: 21 weeks |
| GBM | | 2001) | | |
| #202; NWC; | Aug. 28, 2001- | Depression; Ear | Oct. 24, 2001 | AR25 headache/sinusitis. |
| 45yoF; | Sep. 1, 2001; | disorder; | PFS: 9 Weeks | Died Dec. 23, 2001 |
| R Fronto- | 0.25 μg | Fatigue; | Based on | Survival: 17 weeks |
| temporal GBM | | Headache; | Clinical | |
| | | Sensory | Evidence | |
| | | neuropathy | | |
| #104; TMW | Aug. 29, 2001- | Amnesia; Ataxia; | Sep. 26, 2001 | Post-op 2 catheters used |
| 38yoF; | Sep. 2, 2001; | Headache; | PFS: 5 Weeks | tolerated well. Patient |
| L Fronto- | 0.25 μg | Incoordination; | | replaced in enrollment b/c |
| parietal GBM | | Sensory | | did not receive enough |
| | | neuropathy; | | pre-infusion drug |
| | | | | Died Mar. 11, 2002 |
| | | | | Survival: 28 Weeks |
| #105; S-C | Nov. 8, 2001- | Sensory | Dec. 31, 2001 | Tissue Enhancement (PD |
| 51yoF; | Nov. 12, 2001; | Neuropathy | PFS: 8 Weeks | vs. Drug) |
| GBM | 0.25 μg | | | Last Follow-up: Sep. 27, 2002 |
| | | | | Survival: 47+ Weeks |
| #203; JWS | Nov. 13, 2001- | — | Feb. 22, 2002 | Tissue Enhancement (PD |
| 46yoM; | Nov. 17, 2001; | | PFS: 15 Weeks | vs. Drug) |
| GBM | 0.25 μg | | | Resected Feb. 25, 2002. |
| | | | | Last Follow-up: Sep. 27, 2002 |
| | | | | Survival: 46+ Weeks |

Cohort #3: 1.0 μg/mL Pre-Resection; 0.25 μg/mL Post-Resection

| | | | | Related AEs | | | | | |
|---|---|---|---|---|---|---|---|---|---|
| Patient #; | | Biopsy/ | | Grade ≧ 2 | | | Related AEs | | |
| Initials; Age; | Date of | Catheter | Infusion #1 | and SAEs: | Pathol- | Infusion #2 | Grade ≧ 2 and | Date of | |
| Sex; Dx | Diagnosis | Date | Date; Dose | Infusion 1 | ogy | Date; Dose | SAEs: Infusion 2 | Progression | Comments |
| #301; MJS | Mar. 14, | Feb. 8, | Feb. 9, 2002- | — | 1.0 cm | Feb. 16, 2002- | Pulmonary | Jul. 24, 2002 | Last Follow-up: |
| 69yoF; | 2000 | 2002 | Feb. 11, 2002; | | necrosis | Feb. 20, 2002; | Emboius (SAE, | PFS: | Sep. 27, 2002 |
| GBM | | | 1.0 μg | | | 0.25 μg | Apr. 21, 2002) | 23 Weeks | Survival: |
| | | | | | | | | | 33+ Weeks |
| #401; RRL | Aug. 21, | Feb. 27, | Feb. 27, 2002- | Speech | — | Mar. 7, 2002- | Facial Paralysis; | Jul. 3, 2002 | Last Follow-up: |
| 57yoM; | 2001 | 2002 | Mar. 1, 2002; | Disorder | | Mar. 10, 2002; | Dehydration | PFS: | Sep. 27, 2002 |
| Grade 3 AA | | | 1.0 μg | | | 0.25 μg | (SAE, Apr. 25, | 18 Weeks | Survival: |
| | | | | | | | 2002) | | 30+ Weeks |
| #402; RKW | Mar. 24, | Mar. 21, | Mar. 22, 2002- | Aphasia; | — | Apr. 1, 2002- | CSF leakage f/u | Jun. 21, | Last Follow-up: |
| 49yoM; | 1996 | 2002 | Mar. 24, 2002; | Headache; | | Apr. 4, | (SAE, Mar. 30, | 2002 PFS: | Sep. 27, |
| Anaplastic | | | 1.0 μg | CSF | | 2002; 0.25 μg | 2002); Headache; | 13 Weeks | 2002 |
| Oligo- | | | | Drainage r/t | | | Pneumocephalus | | Survival: |
| dendroglioma | | | | (SAE, Mar. | | | (SAE, Apr. 10, | | 27+ Weeks |
| | | | | 30, 2002); | | | 2002); | | |
| | | | | Seizure; | | | Meningitis (SAE, | | |
| | | | | Sensory | | | Apr. 15, 2002); | | |
| | | | | Neuropathy | | | Cranlotomy Flap | | |
| | | | | | | | Edema (SAE, | | |
| | | | | | | | Apr. 23, 2002); | | |
| | | | | | | | Pulmonary | | |
| | | | | | | | Embolus (SAE, | | |
| | | | | | | | May 11, 2002) | | |
| #106; HLW | Apr. 9, | Mar. 26, | Mar. 27, 2002- | — | — | Apr. 4, 2002- | Headache; Motor | — | Last Follow-up: |
| 52yoM; | 2000 | 2001 | Mar. 29, 2002; | | | Apr. 8, 2002; | Neuropathy; | | Sep. 27, 2002 |
| Anaplastic | | | 1.0 μg | | | 0.25 μg | Seizure: Sensory | | Progression |
| Oligo- | | | | | | | Neuropathy; | | |
| astrocytoma | | | | | | | Broken Leg | | 26+ Weeks |
| | | | | | | | (SAE, | | |
| | | | | | | | Aug. 21, 2002) | | |

Cohort #4: 2.0 μg/mL Pre-Resection; 0.25 μg/mL Post-Resection

| Patient #; | | Biopsy/ | | Related AEs | Pa- | | Related AEs | | |
|---|---|---|---|---|---|---|---|---|---|
| Initials; Age; | Date of | Catheter | Infusion #1 | Grade ≧ 2 and | thol- | Infusion #2 | Grade ≧ 2 and | Date of | |
| Sex; Dx | Diagnosis | Date | Date; Dose | SAEs: Infusion 1 | ogy | Date; Dose | SAEs: Infusion 2 | Progression | Comments |
| #107; KJN | Jan. 3, | May 14, | May 15, 2002- | — | — | May 23, 2002- | — | — | Last Fallow-up: |
| 24yoF; | 2002 | 2002 | May 17, 2002; | | | May 27, 2002; | | | Sep. 27, 2002 |
| GBM | | | 2.0 μg | | | 0.25 μg | | | Progression |
| | | | | | | | | | Free Survival: |
| | | | | | | | | | 19+ Weeks |
| #302; PCJ | — | May 17, | May 18, 2002- | Hyponatremia | — | May 25, 2002- | CSF Leakage | Aug. 22, 2002 | Died: |
| 48yoM; | | 2002 | May 20, 2002; | (SAE, Week 1, | | May 29, 2002; | (SAE, Week 6, | PFS: | Sep. 25, 2002 |
| GBM | | | 2.0 μg | May 21, 2002); | | 0.25 μg | Jun. 22, 2002); | 14 Weeks | Survival: |
| | | | | Headache (SAE. | | | Expressive | | 18 Weeks |
| | | | | Week 1, | | | Dysphasia (SAE, | | |
| | | | | May 21, 2002); | | | Week 11, | | |
| | | | | Vomiting (SAE, | | | Jul. 26, 2002) | | |
| | | | | Week 1, | | | | | |
| | | | | May 21, 2002) | | | | | |
| #303; DMD | Oct. 18, | Jun. 5, | Jun. 6, 2002- | — | — | Jun. 13, 2002- | — | Aug. 6, 2002 | Last Follow-up: |
| 43yoM; | 2001 | 2002 | Jun. 8, 2002; | | | Jun. 17, 2002; | | PFS: | Sep. 27, 2002 |
| GBM | | | 2.0 μg | | | 0.25 μg | | 9 Weeks | Survival: |
| | | | | | | | | | 16+ Weeks |

Cohort #5: 0.5 μg/mL Post-Resection

| Patient #; | | Resection/ | Post Resection | | | |
|---|---|---|---|---|---|---|
| Initials; Age; | Date of | Catheter | Infusion | Infusion Related AEs Grade ≧ 2 and | Date of | |
| Sex; Dx | Diagnosis | Data | Date; Dose | SAEs | Progression | Comments |
| #204; CAM | Apr. 3, | Jul. 24, 2002 | Jul. 26, 2002- | Pulmonary Embolism (SAE, Week 2, | Aug. 19, 2002 | Last Follow-up: Sep. 27, 2002 |
| 60yoM; | 2002 | | Jul. 30, 2002; | Jul. 31, 2002); Deep Vein Thrombosis | PFS: 3 Weeks | Survival: 9+ Weeks |
| GBM | | | 0.5 μg | (SAE, Week 2, Aug. 2, 2002) | | |
| #205; LJP | Jan. 8, | Jul. 29, 2002 | Jul. 31, 2002- | Thrombosis (SAE, Week 2, Aug. 8, 2002); | — | Last Follow-up: Sep. 27, 2002 |
| 56yoM; | 2002 | | Aug. 4, 2002; | Hemiparesis (SAE, Week 8, Sep. 17, 2002) | | Progression Free Survival: 9+ |
| GBM | | | 0.5 μg | | | Weeks |
| #108; J-V | Apr. 3, | Jul. 29, 2002 | Jul. 31, 2002- | — | — | Last Follow-up: Sep. 27, 2002 |
| 47yoM; | 2002 | | Aug. 4, 2002; | | | Progression Free Survival: 9+ |
| GBM | | | 0.5 μg | | | Weeks |

### EXAMPLE 7

In another clinical study intracerebral injection of IL 13-PE38QQR is accomplished using escalating infusion duration from 4 days (51.8 mL) to a maximum of 7 days (90.7 mL), to identify a MTD based on infusion duration; infusion rate held constant at 540 mL/hr (total) as follows:

| | | Duration | | |
|---|---|---|---|---|
| Dose level | Conc. (μg/μL) | (Days) | Total Dose (μg) | Increment (%) |
| 1 | .05 | 4 | 25.9 | — |
| 2 | .05 | 5 | 32.4 | 25 |
| 3 | .05 | 6 | 38.9 | 20 |
| 4 | .05 | 7 | 45.4 | 16.7 |

A second protocol is employed in which concentration escalated from 1.0 a maximum of 4.0 mg/mL (assuming 7-day infusion) to identify a MTD based on ion; infusion rate held constant at 540 mL/hr (total) as follows:

| Dose level | Conc. (μg/μL) | Total Dose (μg) | Increment (%) |
|---|---|---|---|
| 1 | 1.0 | 90.7 | 100 |
| 2 | 2.0 | 181.4 | 100 |
| 3 | 3.0 | 272.2 | 50 |
| 4 | 4.0 | 362.8 | 33 |

This study currently is at dose level 2, and data generated to date are presented lowing two pages:

Cohort #1: 0.5 μg/mL × 4 Days

| Patient #; | | Biopsy/ | | | Related AEs | | | |
|---|---|---|---|---|---|---|---|---|
| Initials; Age; | Date of | Catheter | Pre-resection | Date of | Grade ≧ 2 | | Date of | |
| Sex; Dx | Diagnosis | Date | Infusion Date | Resection | and SAEs | Pathology | Progression | Comments |
| 0110-1101; | Apr. 28, 1992 | Jul. 30, 02 | Jul. 31, 2002 | Aug. 13, 2002 | Seizure | — | — | — |
| JJK; 48 yoF; | | | | | (Grade 2); | | | |
| Anaplastic | | | | | Headache | | | |
| Glioma | | | | | (Grade 2) | | | |
| 0108-1102; | Aug. 8, 2001 | Aug. 5, 2002 | Aug. 6, 2002 | Aug. 19, 2002 | — | — | — | — |
| B-C; 56 yoM; | | | | | | | | |
| Glioblastoma | | | | | | | | |
| 0108-1103; | Jul. 9, 2001 | Aug. 12, 2002 | Aug. 13, 2002 | Aug. 26, 2002 | — | — | — | — |
| Y-E; 54 yoM; | | | | | | | | |
| Glioblastoma | | | | | | | | |
| Multipforme | | | | | | | | |

Cohort #2: 0.5 μg/mL × 5 Days

| Patient #; | | Biopsy/ | Pre-resection | | | | | |
|---|---|---|---|---|---|---|---|---|
| Initials; Age; | Date of | Catheter | Infusion | Date of | Related AEs | | Date of | |
| Sex; Dx | Diagnosis | Date | Date | Resection | Grade ≧ 2 and SAEs | Pathology | Progression | Comments |
| 0108-1201; | April | Sep. 30, 2002 | Oct. 1, 2002 | Oct. 14, 2002 | — | — | — | — |
| I-P; 35 yoF; | 1999 | | | Projected | | | | |
| GBM | | | | | | | | |
| 0108-1202; | March | Sep. 30, 2002 | Oct. 1, 2002 | Oct. 14, 2002 | — | — | — | — |
| Z-F; 61 yoM; | 2002 | | | Projected | | | | |
| GBM | | | | | | | | |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for killing a cell that expresses a receptor for interleukin 13 and that is located in a solid tissue comprising, inserting at least one catheter directly into said solid tissue and administering a cytotoxic agent to said solid tissue under pressure through said catheter into the solid tissue at a flow rate of about 30 μl/h or more to about 1 ml/h for a predetermined period of time such that a portion of said cytotoxic agent contacts a cell that expresses a receptor for interleukin 13 in said solid tissue and kills said cell.

2. A method for treating a solid tumor that contains cells that express a receptor for IL-13 comprising inserting at least one catheter directly into said solid tumor and administering a cytotoxic agent to said solid tumor under pressure through said catheter into the solid tumor at a flow rate of from about 30 μl/h to about 1 ml/h for a predetermined period of time such that a portion of said cytotoxic agent contacts a cell that expresses a receptor for interleukin 13 in said solid tumor and kills said cell.

3. A method for treating a solid tissue tumor that contains cells that express a receptor for IL-13 comprising inserting at least one catheter directly into solid tissue in proximity to a tumor that contains cells that express a receptor for IL-13 and administering a cytotoxic agent under pressure through said catheter toward said tumor at a flow rate of from about 30 μl/h to about 1 ml/h for a predetermined period of time such that apportion of said cytotoxic agent contacts a cell that expresses a receptor for interleukin 13 in said solid tumor and kills said cell.

4. The method of any of claims 1-3, wherein the cytotoxic agent comprises a portion of IL-13 that binds to an IL-13 receptor.

5. The method of any of claims 1-3, wherein the cytotoxic agent comprises a portion of IL-13 that binds to an IL-13 receptor fused to a toxin.

6. The method of any of claims 1-3, wherein the cytotoxic agent is IL13-PE38QQR.

7. The method of any of claims 1-3, wherein said step of inserting at least one catheter directly into said solid tissue and administering a cytotoxic agent to said solid tissue is repeated.

8. The method of any of claims 1-3, further comprising resecting said tissue or said tumor.

9. The method of any of claims 1-3, where said cell is within a tumor.

10. The method of claim 9, wherein said tumor is a glioma.

11. The method of claim 9, wherein said tumor is a brain cancer tumor, or a brain stem cancer tumor.

12. A method for killing a cell that expresses a receptor for interleukin 13 and that is located in a solid tissue comprising, inserting at least one catheter directly into said solid tissue and administering about 1 μg or more to about 1 mg IL 13-PE38QQR to said solid tissue under pressure through said catheter into the solid tissue in a predetermined period of time such that a portion of said IL13-PE38QQR contacts a cell that expresses a receptor for interleukin 13 in said solid tissue and kills said cell.

13. The method of claim 12, wherein about 2 μg or more to about 600 μg IL13-PE38QQR is administered to said solid tissue.

14. The method of claim 12, wherein about 4 μg or more to about 400 μg IL13-PE38QQR is administered to said solid tissue.

15. The method of claim 12, wherein about 4 μg or more to about 100 μg IL13-PE38QQR is administered to said solid tissue.

16. The method of claim 12, wherein about 5 μg or more to about 50 μg IL13-PE38QQR is administered to said solid tissue.

17. The method of claim 12, wherein said step of inserting at least one catheter directly into said solid tissue and administering a cytotoxic agent to said solid tissue is repeated.

18. The method of claim 8 or 17 further comprising resecting said tissue or said tumor.

19. The method of claim 8 or 17, where said cell is within a tumor.

20. The method of claim 19, wherein said tumor is a glioma.

21. The method of claim 19, wherein said tumor is a brain cancer tumor, or a brain stem cancer tumor.
